(19) United States
(12) Patent Application Publication
(10) Pub. No.: US 2021/0251661 A1
(43) Pub. Date: Aug. 19, 2021

(54) CONTINUOUS COMPRESSION DEVICE FOR BONE

(71) Applicants: David B. KAY, Akron, OH (US); Ian KAY, Fairlawn, OH (US); Andrew LEITHER, Akron, OH (US)

(72) Inventors: David B. KAY, Akron, OH (US); Ian KAY, Fairlawn, OH (US); Andrew LEITHER, Akron, OH (US)

(21) Appl. No.: 17/252,064

(22) PCT Filed: Jun. 14, 2019
(86) PCT No.: PCT/US2019/037267

§ 371 Date: Dec. 14, 2020

Related U.S. Application Data
(60) Provisional application No. 62/685,024, filed on Jun. 14, 2018.

(51) Int. Cl. A61B 17/68; A61B 17/72; A61B 17/80

(52) U.S. Cl. CPC A61B 17/68; A61B 17/7225; A61B 2017/681; A61B 17/8023; A61B 17/8004

(57) ABSTRACT

There is disclosed devices and methods for providing continuous bone compression for bone healing. The devices can include both intermedullary and exoskeletal devices. The devices are “tunable”, or specific to, the quality of the bone.

### BACKGROUND OF THE INVENTION

The invention relates generally to orthopedic medical devices and methods, and more specifically, to orthopedic implant devices and methods for applying continuous compression for bone healing.

In the past, surgical optimization for bone healing was often predicated on bone being a static material. However, bone is living, dynamic system and constantly changes based on a variety of biologic and mechanical factors. For example, bone resorption typically occurs at the interface of two bone surfaces. In particular, this is the case, when the bone is incorporated into an orthopedic construct that includes the bone, fixation members, such as screws, and a scaffold member, such as a plate. This bone resorption changes the construct mechanics for example as the screw location such that a less than optimal construct evolves over time. This can lead to failure of bone fusion or healing. Conventional means to address this problem is to rely upon advanced elastic materials, such as nitinol and nitinol polymer compounds. While these materials can be structured to provide continuous compression, they present certain disadvantages. For example, they are often brittle, and difficult to shape. They are expensive, and they are not “tunable”, meaning that they are not able to achieve a direction of amount of force that can be tailored to a specific quality of bone, which results in either insufficient compression or so much compression that the implants can damage the bone, leading to bone necrosis, inflammation and failure of the bone to unite.

### SUMMARY OF THE INVENTION

In one embodiment, the invention is directed to an intramedullary bone continuous compression device, including (a) an external sleeve having inner and outer surfaces and a plurality of openings extending from the outer surface through the inner surface and (b) an internal compression mechanism contained within the external sleeve and configured to apply a compressive force, (which is preferably in the direction of a long axis of the sleeve) to screws implanted in one or more bones or separate pieces of a bone. The internal compression mechanism includes an elongated rod extending within the external sleeve and operatively engaged (i.e., so as to provide a resilient engagement within the sleeve) with an elastic member (i.e., a member that provides an elastic force). Here, the term “intramedullary” is used to indicate that the compression device can reside fully or partially within the intramedullary portion of a bone, and preferably is configured to provide a friction fit along the length of a bone within this soft internal bone portion. Accordingly, the shape of the device in cross-section can be round, including oval or circular so as to form a cylinder or tapered cylinder in three dimensions, with or without splines or even corners that help to hold the device from moving, such as rotating within the intramedullary cavity.

In another embodiment, the invention is directed to an extramedullary continuous compression device (meaning an implant such as a plate or plate assembly that is configured to reside on the surface of a bone), including a) at least one plate having at least one aperture therein; and (b) an elastomeric or metal member in operative engagement with the plate or plate assembly and configured to apply a compressive force directly or indirectly to one or more bones or separate pieces of a bone. As used herein, “plate” refers to a device that generally has a first surface with a second spaced surface that at least for a portion, generally corresponds to the topography of the first surface such that the through thickness is the same at more than one location of the “plate.” The plate may include a radius in one or more directions, for example across the width of the bottom surface of the plate.

In this embodiment, the plate assembly has a longitudinal axis and includes a first plate member and a second telescoping plate member that engages an internal channel within the first plate member. Each of the plate members includes a fixation member that extends downward, and nominally normal to the medial plane of the plate assembly. Preferably, the fixation member, i.e. as screw, is a locking fixation member which is fixed in its orientation relative to the plate and/or plate aperture that holds it. The plate assembly further includes an elastic element which elastically couples the first and second plate member and a deployment device which can be activated to cause the elastic element to close the second plate member relative to the first plate member and reduce the length between fixation members in the first and second plate members. The elastic element can be an elastic ring, an elastic cable, and retractable ring members that may have spring qualities. It is preferable that the elastic element applies the compressive force in a balanced manner on two sides of the longitudinal axis of the plate assembly. The deployment device can be a rotatable cam member, or a spacer block, or can comprise a lock washer ring in a screw slot. The rotatable cam member has the same advantage as the threaded insert that limits the axial length in the intermedullary device, in that it enables a continuously variable control of the compression throughout the rotation of the cam from the greatest to the smallest diameter. The elastic element can encircle the peripheries of the first and second plate members, or can reside in a groove or channel in the plate members, or can be within the channel in the first plate member for the second plate member and can be captured on a boss on each of the two respective plate members.

In a further embodiment, the invention is directed to a method of providing continuous compression for bone healing including the step of inserting the intermedullary device of into the medullary canal of a bone segment, and of using the device of the invention to achieve either a constant or a variable compressive force in a direction along the length of the bone.

In a still further embodiment, the invention is directed to a method of providing continuous bone compression for bone healing including the step of providing an exoskeletal or scaffold member onto a bone segment in an exoskeletal arrangement or scaffold construct in an expanded state and using a deployment device to cause the plate assembly to shorten along the longitudinal axis so as to apply a compressive force between fixation members which extend into bone away from the second plate member relative to fixation members which extend into bone away the first plate member.

In a further embodiment, the invention is related to a compression device having a first externally threaded member which is in axial alignment with a second member having a torque driving surface. The first member is further capable of rotation cooperation about that axis with the second member. In addition, there is an elastic element which can exert a force in the direction of the axis on the first and/or the second member.

The following description and annexed drawings set forth in detail certain illustrative aspects and implementations of the invention. These are indicative of but a few of the various ways in which the principles of the invention may be employed.

### BRIEF DESCRIPTION OF THE DRAWING FIGURES

FIG. 1 is an exploded perspective view of an intramedullary bone compression device in accordance with an aspect of the invention.

FIG. 2(a) is a side view of the device of FIG. 1;

FIG. 2(b) is a cross-section of the device of FIG. 1 taken along line 2-2;

FIG. 3 is a detail of the torque receiving recess of the device shown in FIG. 2;

FIG. 4(a) is a top perspective view of a plate component of a linear bone compression device in accordance with one aspect of the invention;

FIG. 4(b) is a side view of a fixation member for use with the plate component of FIG. 4(a);

FIG. 4(c) is a top view of a linear bone compression device assembly of FIG. 4(a);

FIG. 4(d) is side cross section taken through line 4-4 of FIG. 4(c);

FIG. 4(e) is top exploded perspective of the assembly of FIG. 4(a);

FIG. 5(a) is an exploded top side perspective view of a further embodiment of a bone compression device in accordance with an aspect of the invention;

FIG. 5(b) is a top view of the bone compression assembly of FIG. 5(a);

FIG. 5(c) is a side perspective view of the bone compression assembly of FIG. 5(a);

FIG. 6(a) is a top side perspective and partially exploded view of a further embodiment of a bone compression in accordance with the invention;

FIG. 6(b) is a top side perspective view of the bone compression assembly of FIG. 6(a);

FIG. 7(a) is a perspective view of an extramedullary bone compression device system in an extended position prior to deployment in accordance with an aspect of the invention;

FIG. 7(b) is a view of an extramedullary bone compression device system of FIG. 7(a) in a deployed position;

FIG. 8 is a view of the device of FIG. 7(a) without fixation members;

FIG. 9 is an exploded view of the assembly of FIG. 8;

FIG. 10(a) is a top view of the extramedullary bone compression device of FIG. 7(a) in an extended position;

FIG. 10(b) is a top view of the extramedullary bone compression device of FIG. 7(b) in a compressed position;

FIG. 11(a) is a side view of the extramedullary bone compression device of FIG. 7(a) in an extended position;

FIG. 11(b) is a side view of the extramedullary bone compression device of FIG. 7(b) in a compressed position;

FIG. 12(a) is a bottom view of the extramedullary bone compression device of FIG. 7(a) in an extended position;

FIG. 12(b) is a bottom view of the extramedullary bone compression device of FIG. 7(b) in a compressed position;

FIG. 13(a) is a top side view of the extramedullary bone compression device of FIG. 7(a) in an extended position;

FIG. 13(b) is a top side view of the extramedullary bone compression device of FIG. 7(b) in a compressed position;

FIG. 14(a) is a perspective view of a further embodiment of an extramedullary bone compression device system in an extended position prior to deployment in accordance with an aspect of the invention;

FIG. 14(b) is a view of an extramedullary bone compression device system of FIG. 14(a) in a deployed position;

FIG. 15 is a view of the device of FIG. 14(a) without fixation members;

FIG. 16 is an exploded view of the assembly of FIG. 15;

FIG. 17(a) is a top view of the extramedullary bone compression device of FIG. 14(a) in an extended position;

FIG. 17(b) is a top view of the extramedullary bone compression device of FIG. 14(b) in a compressed position;

FIG. 18(a) is a side view of the extramedullary bone compression device of FIG. 14(a) in an extended position;

FIG. 18(b) is a side view of the extramedullary bone compression device of FIG. 14(b) in a compressed position;

FIG. 19(a) is a bottom view of the extramedullary bone compression device of FIG. 14(a) in an extended position;

FIG. 19(b) is a bottom view of the extramedullary bone compression device of FIG. 14(b) in a compressed position;

FIG. 20(a) is a top side view of the extramedullary bone compression device of FIG. 14(a) in an extended position;

FIG. 20(b) is a top side view of the extramedullary bone compression device of FIG. 14(b) in a compressed position;

FIG. 21 is a top perspective view of a second version of the extramedullary bone compression device of FIG. 14(a) in an extended position;

FIG. 21 is a bottom perspective view of a second version of the extramedullary bone compression device of FIG. 14(a) in an extended position;

FIG. 23 is a top perspective view of a further embodiment of an extramedullary bone compression device system in accordance with an aspect of the invention;

FIG. 24 is a top exploded view of the assembly of an extramedullary bone device of FIG. 23;

FIG. 25 is a side top exploded view of the assembly of an extramedullary bone device of FIG. 23;

FIG. 26 is a top perspective view of a second version of the extramedullary device of FIG. 23;

FIG. 27 is a top exploded view of the assembly of an extramedullary bone device of FIG. 26;

FIG. 28 is a detail of the compression element channels of FIG. 26;

FIG. 29 is a detail of the compression element channels of FIG. 26 following a swaging procedure;

FIG. 30 is a perspective view of a further embodiment of an extramedullary bone compression device system in an extended position prior to deployment in accordance with an aspect of the invention;

FIG. 31 is a top view of a first plate member of FIG. 30;

FIG. 32 is a side view of the plate member of FIG. 31;

FIG. 33 is a bottom view of the plate member of FIG. 31;

FIG. 34 is a top view of a second plate member of FIG. 30;

FIG. 35 is a side view of the plate member of FIG. 34;

FIG. 36 is a bottom view of the plate member of FIG. 34;

FIG. 37 is an exploded top perspective of the assembly of FIG. 30;

FIG. 38 is an exploded bottom perspective of the assembly of FIG. 30;

FIG. 39 is a first detail of the compression element assembly of FIG. 30;

FIG. 40 is a second detail of the compression element assembly of FIG. 30;

FIG. 41 is a perspective view of a further embodiment of an extramedullary bone compression device system in accordance with an aspect of the invention;

FIG. 42 is a top view of a first plate member of FIG. 41;

FIG. 43 is a side view of the plate member of FIG. 41;

FIG. 44 is a cross section view of the plate member of FIG. 41 taken at line I-I;

FIG. 45 is a cross section view of the plate member of FIG. 41 taken at line J-J;

FIG. 46 is a top perspective exploded view of the assembly of FIG. 41;

FIG. 47 is a top view of a second version of the extramedullary bone compression device system shown in FIG. 41;

FIG. 48 is a top side view of the extramedullary bone compression device assembly of FIG. 47;

FIG. 49 is a side view of the extramedullary bone compression device assembly of FIG. 47;

FIG. 50 is a cross- section of the extramedullary bone compression device assembly of FIG. 49 taken at line K-K;

FIG. 51 is a top perspective exploded view of the extramedullary bone compression device assembly of FIG. 47;

FIG. 52 is a top perspective view of a further embodiment of the extramedullary bone compression device system in accordance with the present invention;

FIG. 53 is a side view of the extramedullary bone compression device assembly of FIG. 53;

FIG. 54 is a cross- section of the extramedullary bone compression device assembly of FIG. 52 taken at line L-L;

FIG. 55 is an end view of the extramedullary bone compression device assembly of FIG. 53; and

FIG. 56 is a top perspective exploded view of the extramedullary bone compression device assembly of FIG. 53.

### DETAILED DESCRIPTION OF THE INVENTION

The invention presents multiple “tunable” methods that will allow for directed scalable continuous constant, or variable compression of bone segments to facilitate for bone healing in fracture repair, bone osteotomies and bone fusions. Similarly, these devices can be used in soft tissue repair to address the inherent creep that occurs to ligaments and tendons that are used in reconstruction.

The ideal conditions for bone healing are well documented and include compression that allows for minimal micromotion; reduction in torsion at the bone surface interface for fracture healing, reconstruction osteotomies and fusions; minimal surgical disruption to the blood supply so as to avoid or reduce disvascular bone and cause further inflammation to the healing site; and fixation that is overly rigid and creates stress shielding and prevents bone healing.

The invention describes both endoskeletal and exoskeletal methods to create continuous compression in bone that is physiologically axially loaded and bone that is not axially loaded in use. Physiological axial bone loading is seen in the femur and the tibia during standing, for example. The axial loading, that occurs with weight bearing, is leveraged using intramedullary rod fixation that has a fixed end and the opposite site of fixation is allowed to slide in one plane. However, if there is no weight bearing the loading does not occur. In addition, the loading to the bone surface varies based upon the individual patient's abilities to load the bone. Physiologically non-axially loaded bones, such as in the midfoot, tend to undergo shear with weight bearing and may be a causative factor in the reported high incidence of non-healing bone.

FIG. 1 illustrates an exemplary intermedullary bone compression device 10 in accordance with an embodiment of the invention. The device 10 includes an external sleeve, shown as a cylinder 12 having inner 9 and outer surfaces 11 and a plurality of openings 13 extending from the outer surface of the cylinder 12 through to the inner surface of the cylinder 12. The device 10, further includes an internal compression mechanism 18 contained within the external sleeve 12 and configured to apply a compressive force to one or more bones or separate pieces of a bone. Aperture 14 extends from an outer surface 11 of external cylinder 12 to an inner surface 9 of external cylinder 12. Aperture 14 allows for securing of the outer cylinder 12 to a bone or bone segment by means of a fixation member (i.e., a screw) without interference with movement of the internal compression mechanism 18 within the external cylinder 12. Moreover, aperture 14 is elongated or slotted so that device 10 can move relative to the associated fixation member when it is fixed in the bone.

The inner surface of the external sleeve has wider cylindrical openings at the top and bottom of the sleeve which forms stepped portions at either end of the internal through bore adapted to receive an internal compression mechanism. This results in a first annular flange 22 and a second annular flange 25 on the inside of the external sleeve 12.

In FIG. 2(b), a cross-section of device 10 along the line 2-2 illustrates the internal compression mechanism 18 contained within the external cylinder 12. The internal compression mechanism 18 is configured to apply a compressive force to one or more bones or separate pieces of a bone by either pushing or pulling one or more bones or separate pieces of a bone into a compressive arrangement. The internal compression mechanism 18 has a sliding engagement with the internal surface 9 of the external sleeve 12 and extends within the external sleeve 12 on its longitudinal axis. The external sleeve has a mating top portion 8 which is internally threaded to mate with the threads on the internal rod. The top mating portion includes a central hollow that forms an internal should surface which bears against an elastic element 24, and where the rotation of the top portion determines the amount of force that the external cylinder applies to the elastic element 24. At an opposing end, the elastic element (or here shown as two elastic elements) bears against the top surface in a hollow in the inner rod.

The mechanism 18 includes a rod 21 having a threaded first end 26, an elongated central shaft portion 25, and a second tapered end 28. An internal space 23 within the external sleeve 12 receives the outer surface of rod 21. First end 26 of the rod has a first diameter which narrows inwardly an upper annular shoulder 27. A narrower central shaft portion 25 extends longitudinally between the upper shoulder 27 to second end 28. The second end 28 has a second diameter with a second annular shoulder. The diameters of the mechanism 18 will vary, based upon anatomical requirements and patient size. The rod 21 can be formed from one or more metals, such a stainless steel, or titanium or other biocompatible materials, such as PEEK or hydroxyapatite.

The internal mechanism 18 further include an elongated aperture 14, and an elastic member 24. In one embodiment, the elastic member 24 can provide a pre-compressed loading (i.e., a push in the direction of the long axis of the bone) to the bone or bone segment. The elastic member 24 is situated within internal space 23 between the shoulder 27 and the central shaft portion 25 of rod 21, allowing rod 21 to extend through a central opening of the elastic member 24. The elastic member 24 can include a spring, for example, a leaf spring, a coil spring, a Belleville washer, or the like, and is formed from a metal, a metal alloy or an elastomeric material. Such metal or metal alloys can include titanium, or titanium alloy. A first end of the elastic member 24 is affixed to a non-movable anchor point and a second end of the elastic member 24 is affixed to a movable anchor point. The movable anchor point has a primary translational degree of freedom along the central axis of the external cylindrical sleeve 12.

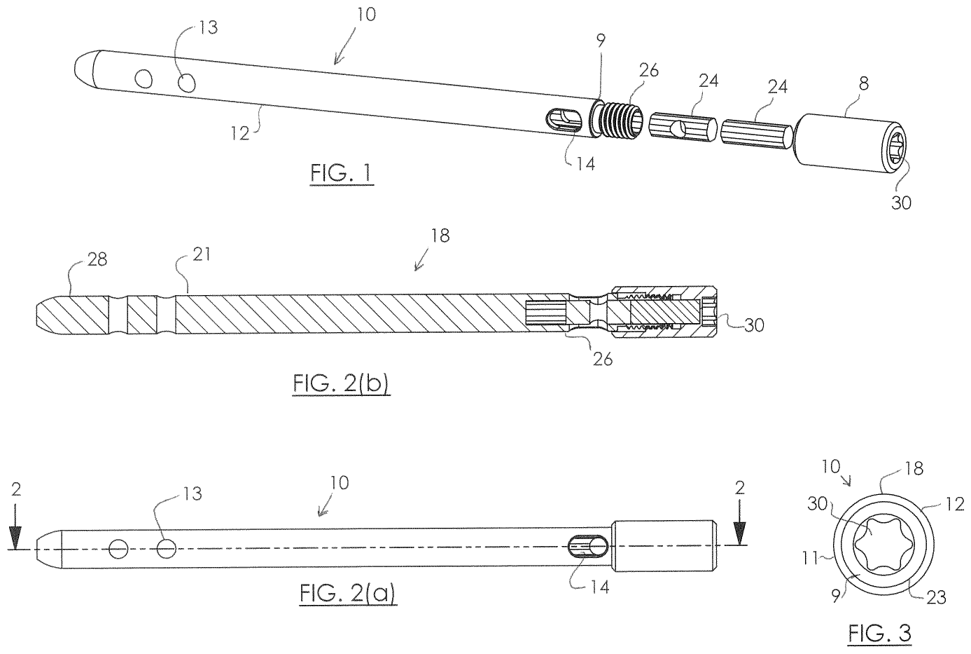

FIG. 3 is a detail of a threaded anchor mount 8 which comprises a torque receiving element 30 of the device 10 shown in FIG. 1-2. The torque receiving element 30 is located in the external cylindrical sleeve 12 in an opposing relationship to the non-moveable anchor point of the elastic member 24 on the external sleeve such that tightening or loosening of the torque receiving element 30 changes the longitudinal location and thus, serves push or pull the rod 21 to compress or expand the elastic member 24 in order to “tune” the force applied so as to provide a desired degree of compression. The element 30 is operatively engaged with the internal compression mechanism 18. The element 30 may be a hexagon, a torx shape, or a modification of a torx shape, i.e. a multilobe shape having from 1 to 10 lobes, and in one embodiment from 4 to 6 pointed sides or lobes.

A first fixation member extends through the intermedullary bone compression device at the aperture 14 and engages the interior surface of the associated bone and a second fixation member extends through the intermedullary bone compression at the aperture 13. Since the device 10 is anchored by means of the fixation member extending through the aperture 14, if a compressive force is applied by the elastic member to push the rod 21, the bone associated with the fixation member 13 will be subjected to a compressive force. Thus, if the element is tightened, the elastic member is compressed and this force is transmitted to the associated bone. Moreover, this force allows for a dynamic situation if bone is resorbed such that the device allows for “trampolining”.

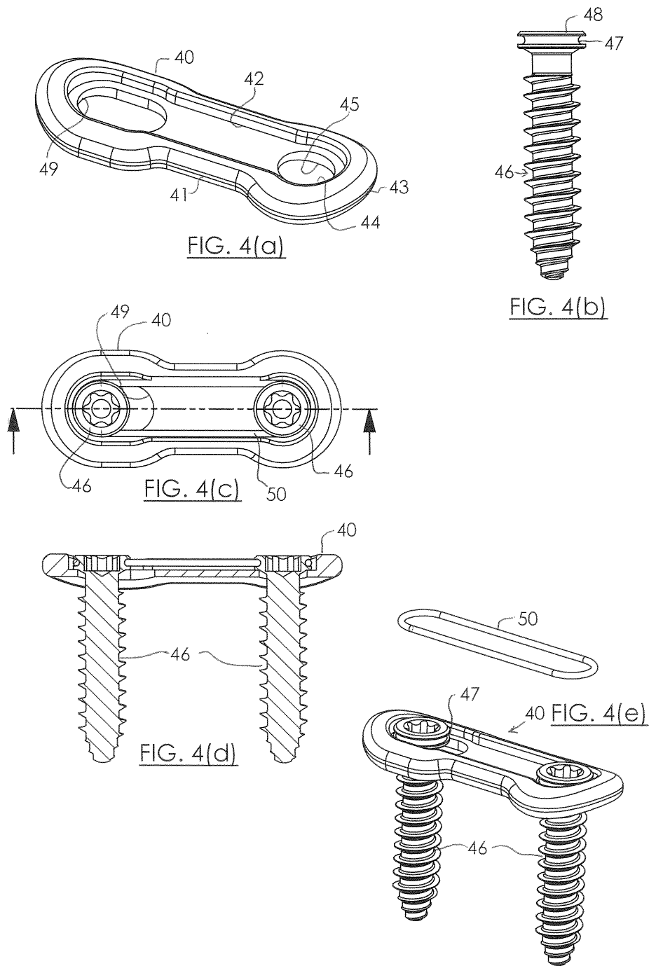

The invention further includes an exoskeletal or extramedullary bone compression device 40. The exoskeletal device includes at least one plate having at least one aperture 45 therethrough. In the device illustrate in FIG. 4(a)-4(e), the device has a recess 42 in a top surface and two through apertures, one, which is round 44, and one which is slotted 49 to accommodate a force along the long axis of the plate member 43. This device is a “two hole” or peanut style device that is configured to accept only two screw members connected by a single bridge member. Thus, the outline consists of a first end having a hole and a second end having a slot which are joined by a bridging middle section, and the plate is configured to minimize the material other than that needed to surround and support the screws and to join them. A suitable fixation member, or screw, 46 is shown in FIG. 4(b). The screw has a threaded portion and a proximal head 48 which includes a groove shaped to accept a looped elastic compression member 50.

The aperture(s) 45 can be configured to accept a screw including having means for locking, such as the provision for internal threads that mate with external threads on the head of a bone screw. Typically, the plate has an outline that is suited for placement on the external surface of a bone, and the plate has a first surface that is curved so as to fit against the bone surface, and a second concentric surface which faces away from the bone with a relatively uniform through thickness defined between the first and the second surfaces. It should be understood, however, that the plate may include raised portions, such as reinforcements, such as about holes for fixation members, like screws.

An elastomeric or metal member 50 is in operative engagement with the plate. The elastomeric or metal member can be wrapped around the screw so as to apply a compressive force to one or more bones or separate pieces of a bone. For example, the elastomeric member, may be an elastic loop or band which loops around a member on the plate, and/or fixation members which fix the plate to underlying bone so as to apply a force between the two members. For example, the plate may include a compression slot having sloped shoulders and a screw that engages the bone through the slot may further include a first end of the elastomeric member and a more typical fixed screw or locking screw may include the second end of the elastomeric member. Alternatively, the plate or fixation member may include brackets for the engagement of the compressive member (i.e. the elastomeric member.) As set forth hereinabove, the plate can be formed from titanium, a titanium alloy or polymeric material.

Referring to FIG. 4(a), there is illustrated a first embodiment 40(a) of the exoskeletal bone compression device 40. In such embodiment, the device 40 includes a plate 41 which has an outline that extends along a long axis, and as is appropriate according to the intended use, may include various rounded features to provide additional material to accommodate an opening or screw hole. The plate 41 further contains two apertures 45, 49 which are configured to accept screws 46 extending from the apertures). The top or bottom surface of the plate includes a recess 42 to form a side edge of increased thickness extending in a vertical direction about apertures 45, 49. An elastomeric or metal member 50 is wrapped around the screw heads and captured in the groove 47 to apply a compressive force to the screws along the axis of the plate 41.

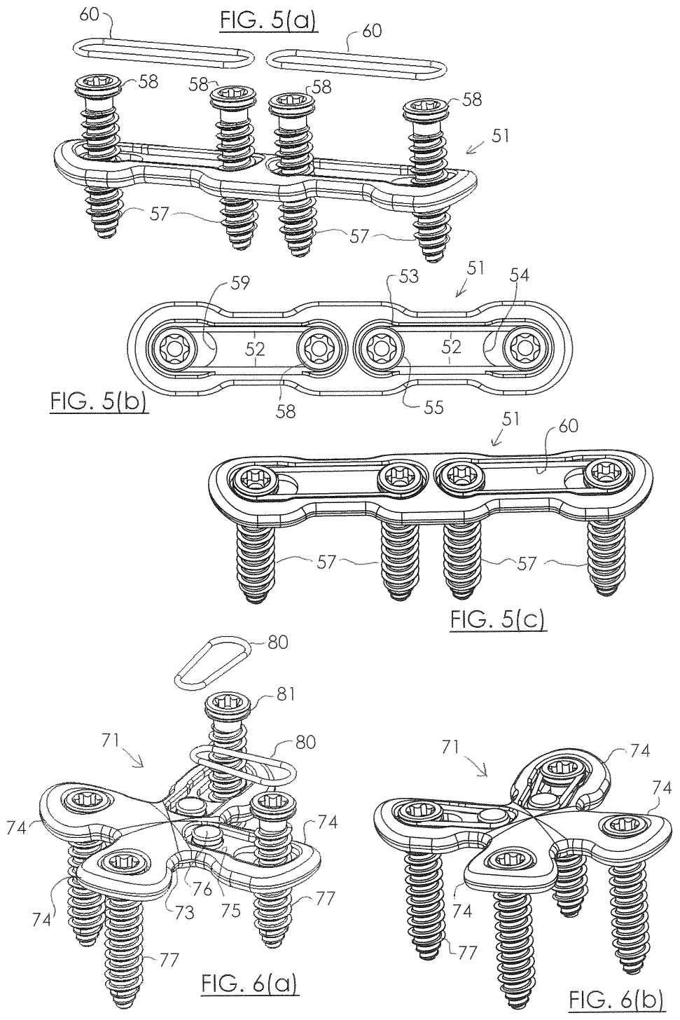

In FIG. 5(a)-5(c) there is illustrated a further embodiment 51 of a linear configuration of the device 40. The embodiment 51 has a plate member 52 that is longer and accommodates additional bone screws (i.e. from 3-8, and preferably 4-6) so as to fix more bone segments together. In this instance the plate has two recesses 52 which are similar in the construction to the device shown in FIG. 4(a), but this time with the slotted holes 59 on the outside of the recesses and the round apertures 53 on the inside so as to form two compression means that are in opposition. The screws 57 also have screw heads having grooves 58 A first elastomeric or metal member 60 extends from screw extending from a first slotted aperture 59 to screw extending from a first round aperture 53. A second elastomeric or metal member 60 extends from screw extending from the second round aperture 53 to the screw extending from the second slotted aperture 59, thereby applying continuous compression toward a transvers axis of the plate and between bone fragments.

In FIG. 6 there is illustrated a still further embodiment of the exoskeletal bone compression device 71. FIGS. 6(a) and 6(b) illustrates a plate 72 having modified X-shaped or cloverleaf configuration in which a central confluence 73 includes two opposing pairs of arms 74 extending therefrom. Each arm 74 includes an aperture to receive a bone screw 77. In the plate illustrate, the top surface of the plate includes two recesses which have lengths that are relatively transverse to each other. These recesses include a post 76 and a slotted aperture and the associated bone screw has a head that includes a groove 81 which accepts an elastic loop 80 to draw compression toward the center of the plate. An elastic loop 80 extends around an outer circumference defined by the screws of the opposing pairs of arms. The loop can be elastomeric or metal.

The invention further includes methods of providing continuous bone compression for bone healing. In a first method utilizing the intermedullary bone compression device 10, the device 10 is inserted into the medullary canal of a bone segment(s) in a surgical procedure. In a second method utilizing the exoskeletal bone compression device 40, the device is applied in an exoskeletal arrangement to a bone segment. In both methods, the amount of compression applied to the bone segment(s) can be made patient specific or specific to the needs of a typical procedure involving this area of the body. This is accomplished by assessing the quality of the bone during the surgical procedure and selecting from varying spring numbers or elastomeric bands of varying durometer and elasticity.

The invention finds particular use in bone segment(s) including the midfoot, the hind foot, the toe or finger phalange(s), the lumbar spine, the pelvis, the hip, the femur, the tibia, the ankle, or the wrist. However, it is further contemplated that the inventive devices can be used in soft tissue repair to address creep that occurs to ligaments and tendons used for reconstruction.

Although the invention has been illustrated and described with respect to one or more implementations, alterations and/or modifications may be made to the illustrated examples without departing from the spirit and scope of the appended claims. In particular regard to the various functions performed by the above described components or structures (assemblies, devices, circuits, systems, etc.), the terms (including a reference to a “means”) used to describe such components are intended to correspond, unless otherwise indicated, to any component or structure which performs the specified function of the described component (e.g., that is functionally equivalent), even though not structurally equivalent to the disclosed structure which performs the function in the herein illustrated exemplary implementations of the invention. In addition, while a particular feature of the invention may have been disclosed with respect to only one of several implementations, such feature may be combined with one or more other features of the other implementations as may be desired and advantageous for any given or particular application. Furthermore, to the extent that the terms “including”, “includes”, “having”, “has”, “with”, or variants thereof are used in either the detailed description and the claims, such terms are intended to be inclusive in a manner similar to the term “comprising”.

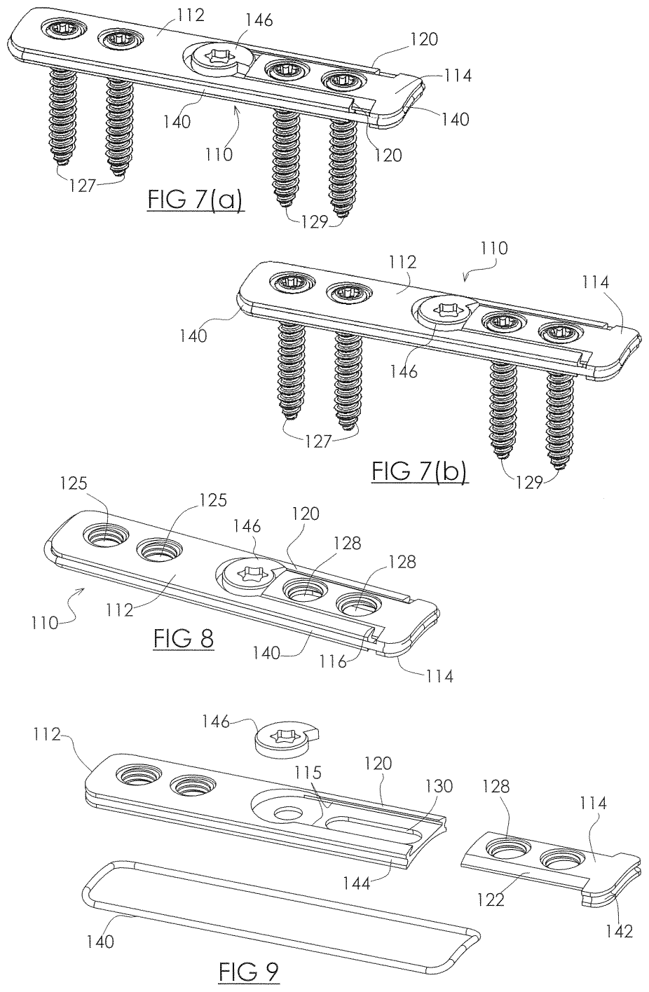

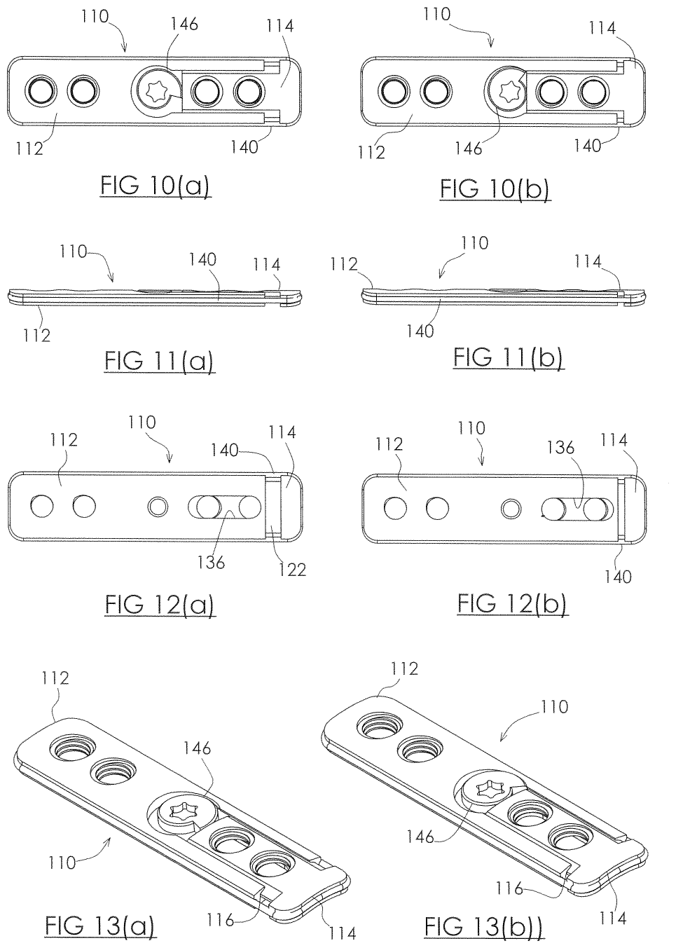

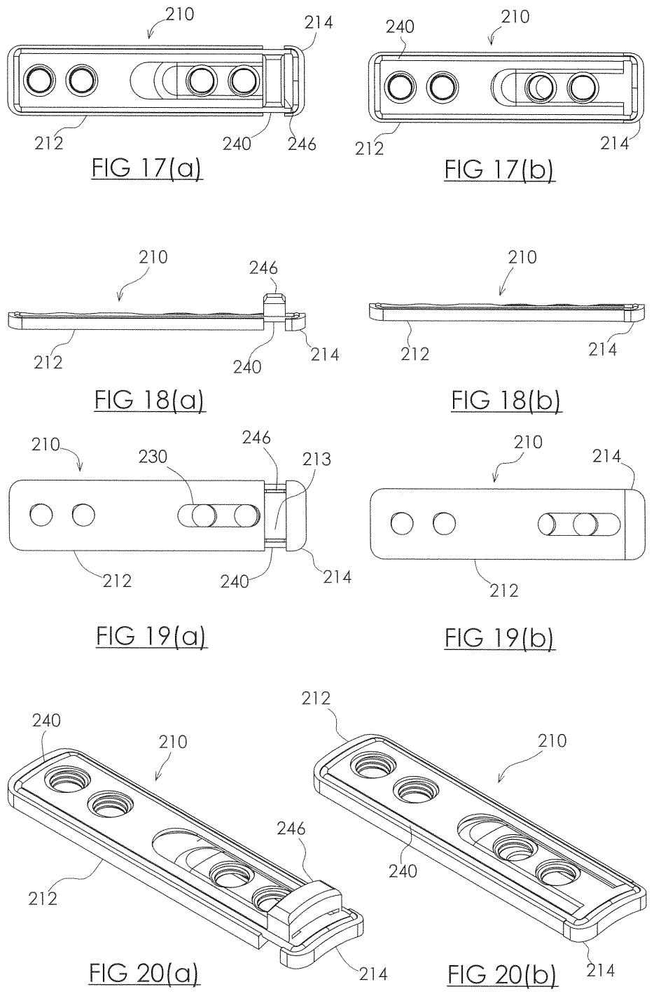

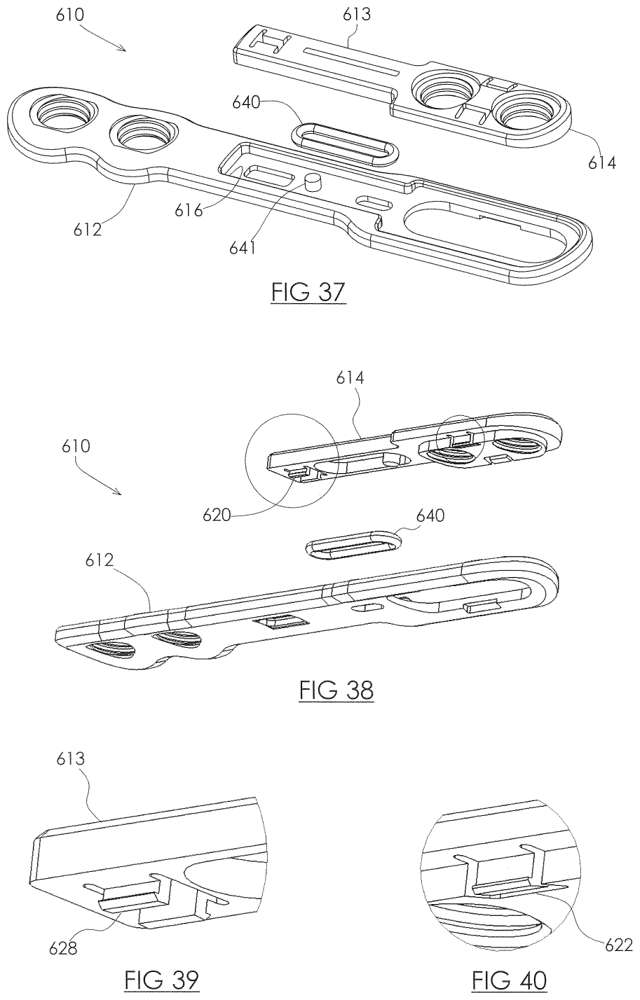

A further embodiment of the extramedullary device of the present invention is illustrated in various versions in FIGS. 7-51, and FIGS. 52-56 illustrate a screw having the compressive attributes of the present invention. FIGS. 7-13(b) show an embodiment of the compressive plate of the present invention 110 which comprises an assembly having a first plate member 112 and a telescoping second plate member 114. The first plate member includes a channel 115 which is clearly set forth in FIG. 9 that is formed by being undercut into the depth of the first plate member, and where the channel has two opposing side brackets 116 which have internal side edges 118 bounded at right angles by top flange members 120. Thus, a housing is formed having bearing surfaces for the sliding insert of the second plate member, which comprises the bottom surfaces of the top flange members 120, the inside surface of the internal side edges 118, and the top surface of the channel 115. Thus, the channel provides extensive bearing surfaces that area axially symmetrical to guide the telescoping of the second plate member so that it is unlikely to jam by becoming axially mis-aligned. The second plate member has a mating extension 122 which has side edges that are formed to mate in a sliding cooperation with the internal side edges of the first plate member channel. The first plate member includes one or more apertures 125, illustrated as threaded locking apertures, and shown in the system of FIGS. 7(a) and 7(b) as including fixed locking screws having a mating threaded head. It should be understood, that this fixation members could include other means of fixation, such as variable locking or non-locking screws. The second plate member similarly includes fixation member apertures 128 which line up with a slot 130 in the channel of the first plate member 112. These apertures 128 receive fixation members 129.

The plate assembly also includes a compressive mechanism which comprises an elastic loop 140 which is housed in grooves 142, 144 in the edges of the first and second plate members so as to compress the plate members together along the long axis of the plate. This compressive mechanism further includes a cam member 146 which has a spiraling outer diameter and where that defines an outer edge member that sits against an end of the second plate member to resist the inward telescoping of the second plate member urged by the elastic loop. The cam member 146 has a threaded engagement with the fist plate member and a top torque driving recess that allows the position of the cam member to be adjusted, and accordingly, the distance of the second plate in the first plate channel.

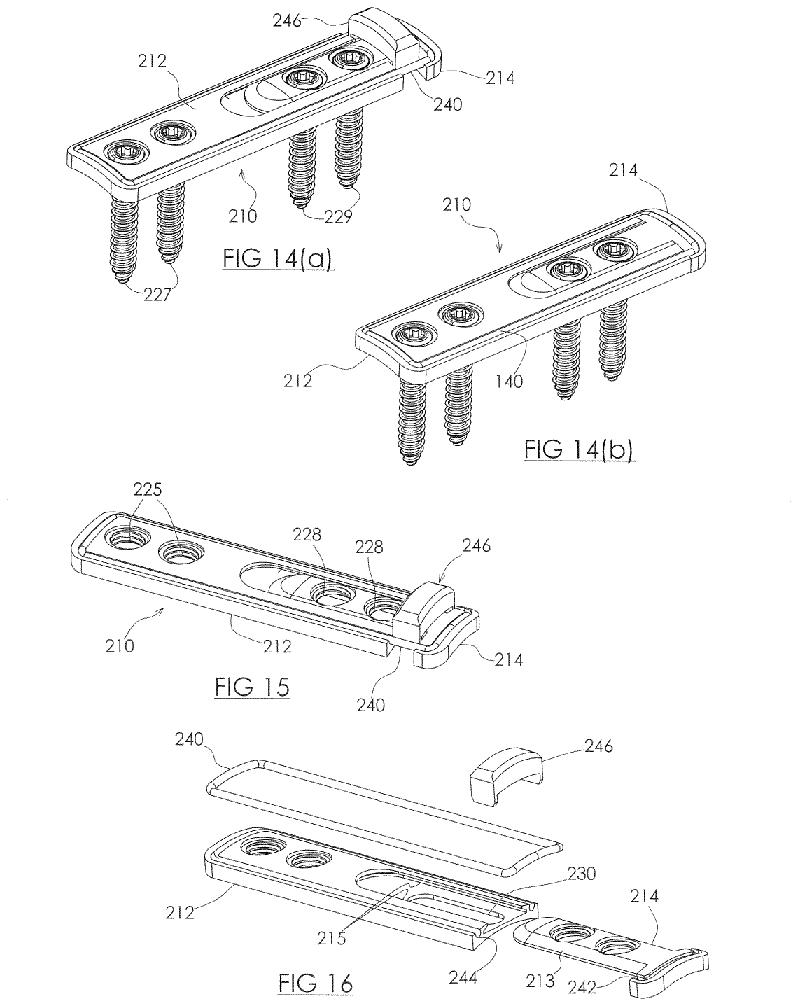

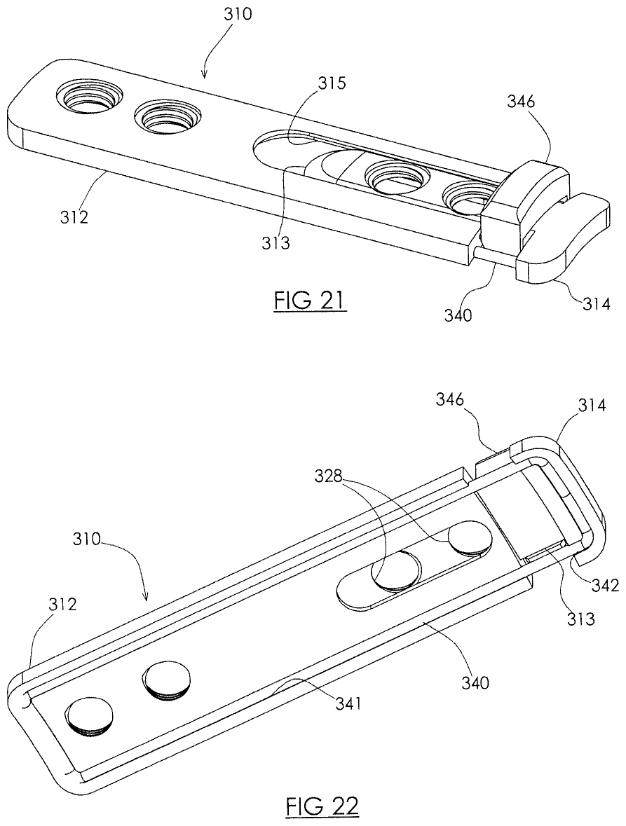

A further version of this plate is shown in FIGS. 14(a) thorough 20(b), and with a different position of the elastic loop in FIGS. 21 and 22. FIGS. 14(a) and 14(b) illustrate a compressive plate assembly 210 having a first plate member 212 with an internal channel 215 that receives an guides a tongue extension 213 of a second plate member 214. The first plate member 212 includes at least one, and preferably 2-4 apertures 225 to receive fixation members, such as screws 227, while the second plate member also includes at least one, and preferably from 2-4 apertures 228 to receive fixation members, such as screws 229. Again, the channel 215 is configured as in the first version includes supporting internal edges and bottom surface and a slot 230 which allows the fixation member 129 to extend through the channel in the first plate member and not interfere with the telescoping of the second plate member relative to the first plate member. Rather than the pivoting cam mechanism, this version of the plate assembly includes a block member 246 which holds the second plate member 214 in a spaced out relative to the first plate member 212. The assembly also includes the compressive mechanism which includes an elastic loop 240 that is received in peripheral grooves 242,244 in the top surfaces of the first and second plate members.

A further version of the compressive plate assembly shown in FIGS. 7(a) through 20(b) is shown in FIGS. 21 and 22. This version of the plate assembly 310 once again includes a first plate member 312 having a support channel 315 that holds a tongue 313 of the second plate member 314 in a telescoping relationship. Once again, the compressive mechanism includes an elastic loop 340 which is held in a tensioned state by a block member 346 that is removed prior to the implantation of the device to cause the elastic loop 340 to apply a force to the second plate member 314, and to the associated fixation members that extend through apertures 328 in the second plate member and through the slot 330 in the bottom surface of the channel 315 in the first plate member 312. In this version, the grooves 342, 344 that house the elastic loop in the first and second plate members is in the bottom (i.e. the surfaces that face toward the bone in use) surfaces of these members.

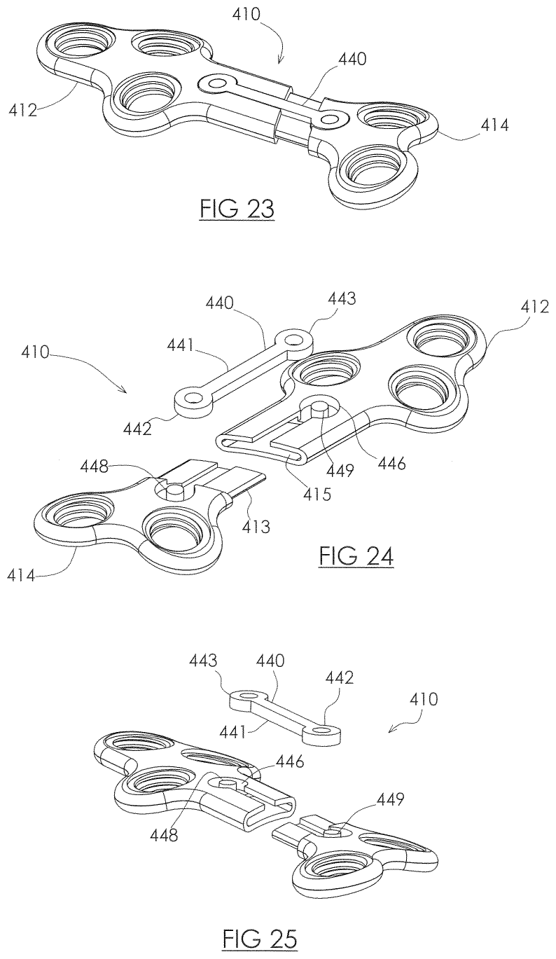

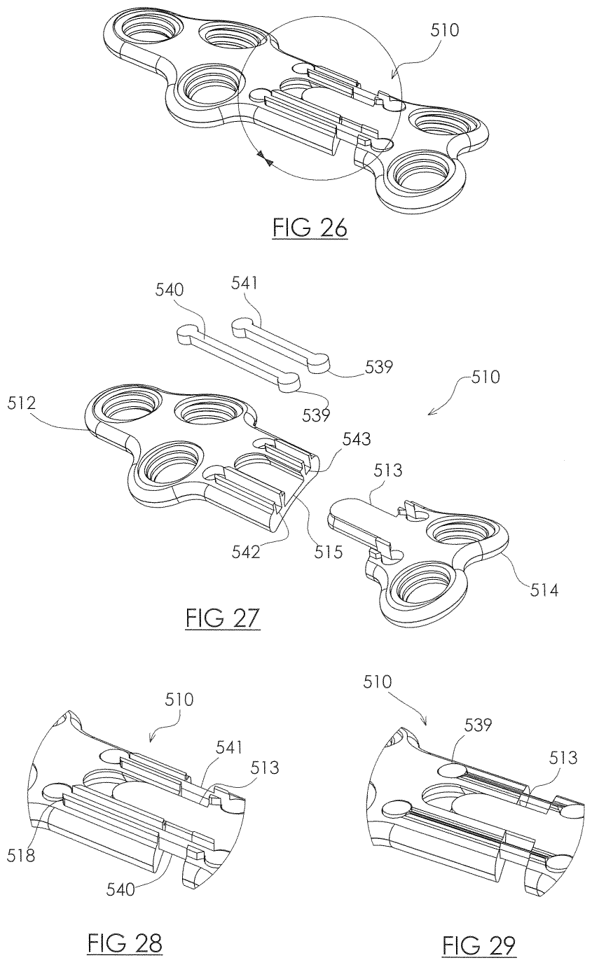

A version of the compressive plate assembly 410 is illustrated in FIGS. 23-25. In this version, the first and second plate members, 412, 414, have a different outline which may better correspond to a different anatomical application. For example, the first plate member terminates in a tri-lobed configuration in which the lobes include apertures for fixation means, and the second plate member has a bi-lobed configuration having apertures for fixation means. The assembly includes a first plate member 412 with a channel 415 that receives and axially supports the tongue 413. Once again, the extension which slides relative to the other plate member is supported in a radially symmetrical way and on the top bottom and sides of the member (i.e. around the axial of movement) so that the movement is less likely to come off-axis and jam the telescoping movement. The compressive element is an elastic dog-bone member 440 that has a long center section 441 which joins a first ring 442 and a second ring 443. The dog-bone 440 is captured in a correspondingly shaped recess 446 in the plate members 412, 414, which also includes a first and second boss 448, 449. FIGS. 26-29 illustrate a slightly different version of this plate assembly configuration 510, again having a first plate member 512 and a second plate member 514, where the second plate member 514 has a tongue 513 that is held in three dimensions in the channel 515 in the first plate member by the bottom surface of a top bracket member, the side edges and the top surface of the bottom of the channel. In this case, the assembly includes two dog-bone elastic members 540, 541, having round stops 539 at each end which are received in two recesses 542, 543 in both of the first plate and second plate members. The recesses have rounded end holds that capture the round stops 539. These recesses are formed having up-right flanges 518 which can be folded or swaged over to capture the elastic members as is illustrated in FIGS. 28 and 29.

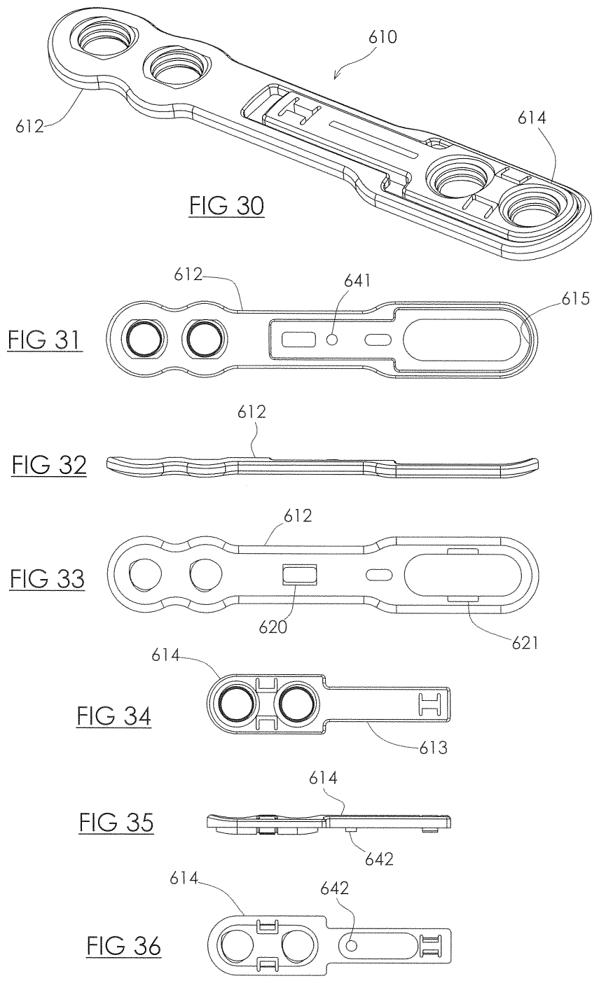

FIGS. 30-40 illustrate yet another plate assembly configuration 610, in which the first plate member 612 has an inset recess 615 in lieu of the channel of the previous versions. Here, the recess is reamed from the surface of the plate, and the second plate member 614 has a tongue member 613 that is supported in a narrowed section 616 of the inset recess 615 and the tongue member 613 includes a set of downwardly extending L-brackets 628 which engage cut-outs 620 on the inset recess to hold the vertical relationship, along with a pair of L-brackets 622 on the back portion of the second plate member which engage cut-outs 621 along the fixation member slot 630 in the inset recess in the first plate member. In this instance, the elastic loop 640 is captured on a first upward facing boss 641 on the inset recess of the first plate member 612 and on a second downward facing boss 642 on the second plate member.

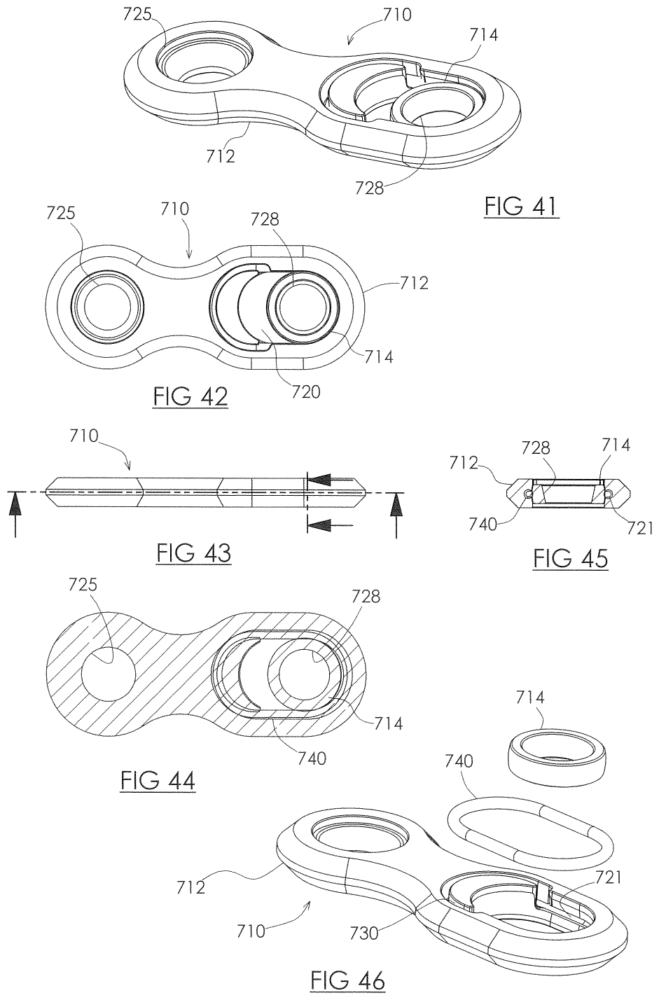

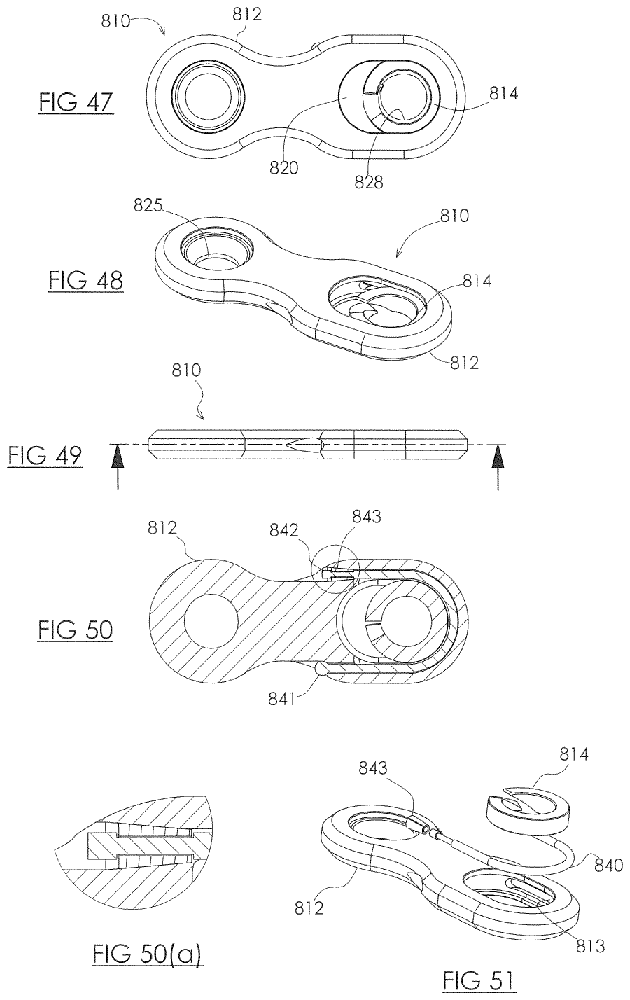

A further version of the plate assembly 710 is shown in FIGS. 41-46 which has a “peanut” plate configuration and in which the first plate member has a locking screw aperture 725 and further an opening 720 and includes a groove 730 that receives one end of an elastic loop 740, and an opening 720 which receives a ring member 714 in lieu of the second plate member. The elastic loop surrounds the ring member exterior surface and fits into a groove 721 in the opening 720. FIGS. 47-51 show another version 810 of this configuration having the first plate member 812 which has the aperture 825 and an opening 820 which received a ring that is shaped to spiral like a lock washer. The elastic loop is replaced by a u-shaped cable 840 having retaining stops 841 on each end, and which reside in a groove 813 in the opening 820 in the first plate member.

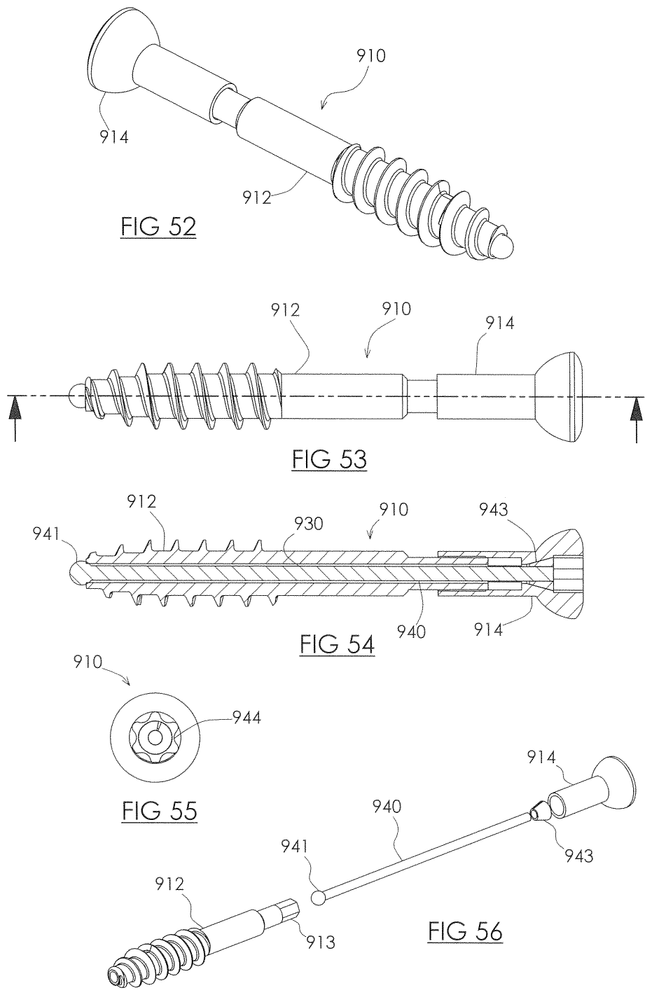

In a further embodiment shown in FIGS. 52-56, the invention is related to a compression device, here a screw, having a first externally threaded member which is in axial alignment with a second member having a torque driving surface. The first member is further capable of rotation cooperation and disengagement about that axis with the second member. In addition, there is an elastic element which can exert a force in the direction of the axis on the first and/or the second member.

In this embodiment, the invention comprises a two part screw member 910 having a first threaded portion 912 that has a post 913 having a torque driving shape at the proximal end, and a central axial cannulation 930. The post 913 is received in a corresponding torque driving recess in a second head screw portion 914 which also has a central cannulation. The post 913 and recess are configured to allow a disengagement of the two parts which is caused by opposing a compressive force on the elastic element which acts on one or both of them. In this case, the elastic element is an elastic cable member 940 is captured in the central cannulation. The elastic cable has rounded stops 941 at either end which hold the cable in the two parts by being captured in the central cannulations. The head screw portion includes a torque driving recess 944 at the end and the head has a larger diameter than the shaft of the threaded portion. In addition, the screw has a length along the axis that is not threaded so that the screw acts like a lag screw. As the screw is tightened into bone, the rear section of the head bears against the bone which tensions the inner elastic member. This tension continues to bear against the bone which is captured between the head and the threads on the distal end of the screw assembly.

In other aspects of the invention, devices and methods include the following.

14. An orthopedic implant having continuously tunable variable compression for a bone or bone segment comprising: a construct defining an axis and having a first member with at least one first member fixation structure capable of fixing the first member to the bone segment, and a second member with at least one second member fixation structure capable of fixing the second member to the bone segment; and a compression mechanism operatively coupled to the first member and to the second member and capable of applying a force to the first member or the second member in the direction of the axis and having a control that adjusts the degree of force by means of the rotation of an associated element, wherein the construct is an orthopedic plate.

15. An orthopedic implant as set forth in 14, wherein the first construct member is a first plate member and the first fixation structure is a first plate member aperture which receives a first plate fixation member and the second construct member is a second plate member and the second fixation structure is a second plate member aperture which receives a second plate fixation member and the compression mechanism is a telescoping relationship between the first plate member and the second plate member subject to a force applied in the direction of the axis by an elastic element and the control is a rotatable cam.

16. An orthopedic implant as set forth in 15, wherein the elastic element is an elastic loop or cable.

17. An orthopedic implant as set forth in 16, wherein the elastic element resides in a groove on a surface of the construct.

18. An orthopedic implant having continuously tunable variable compression for a bone or bone segment comprising: a construct defining an axis and having a first member with at least one first member fixation structure capable of fixing the first member to the bone segment, and a second member with at least one second member fixation structure capable of fixing the second member to the bone segment; and a compression mechanism operatively coupled to the first member and to the second member and capable of applying a force to the first member or the second member in the direction of the axis and having a control that adjusts the degree of force by means of the rotation of an associated element, which is an orthopedic screw having a first screw portion and a second screw head portion.

19. An orthopedic implant as set forth in 18, wherein the first screw member and the second screw member each have a cannulation and the compression mechanism is an elastic cable that is received in the cannulations of the first member and the second member, and the control is a torque driving surface in the head of the screw that is resisted by the bone or bone segment in use.

20. An orthopedic implant as set forth in 19, wherein one of the first screw member or the second screw member have a recess and the other of the first screw member and the second screw member have a post which cooperates with said recess.

21. An intramedullary compression assembly for use in one or more bones or bone segments and comprising: an external sleeve having a central opening extending along an axis and having outer and inner surfaces about the axis and at least two fixation member openings which are not along the axis and extend from the outer surface through the inner surface; an internal compression mechanism contained within the external sleeve central opening and configured to apply a compressive force to the one or more bones or bone segments, the internal compression mechanism comprising a compression member extending in the direction of the axis within the central opening of the external sleeve and which acts to apply a force in the direction of the axis; and at least two fixation members that extend through the openings wherein the force is transmitted to the one or more bone segments by the fixation members.

22. The device of 21, wherein the fixation members are screws.

23. The device of 22, wherein at least one of the fixation members is a locking screw and wherein one of the openings is not circular and is longer than it is wide.

24. The device of 23, wherein the external sleeve is a cylinder and the fixation members each have long axes that intersect the axis of the external sleeve in a perpendicular orientation.

25. The device of 21, wherein the internal compression mechanism resides in the central opening of the external sleeve and forms a sliding engagement with the inner surface of the external sleeve in the direction of the longitudinal axis.

26. The device of 21, wherein the compression member further comprises a rod which has a first end with a first diameter, a central shaft portion, and a second end with a second diameter.

27. The device of 26, wherein the rod has a first shoulder which is an external upper shoulder and a second shoulder which is an external lower shoulder and the central shaft portion extends longitudinally between the upper shoulder of the first end and the lower shoulder at the second end, and the external sleeve central opening has a first shoulder which is an internal upper shoulder and second shoulder which is an internal lower shoulder.

28. The device of 28, wherein an elastic member is situated within an internal space between the external upper shoulder and the central shaft portion of rod and the internal upper shoulder of the central opening.

29. The device of 28, wherein a first end of the elastic member is affixed to an anchor point on the central opening and a second end of the elastic member is affixed to an anchor point on the rod.

30. The device of 21, wherein the anchor point on the rod is movable along a single direction of motion normal to a surface of the bone.

31. The device of 21, wherein the anchor point on the rod has a primary translational degree of freedom along the central axis of the cylinder.

32. The device of 31, wherein the force applied by the compression mechanism on the fixation members can be adjusted in use.

33. The device of 32, wherein the compression mechanism comprises an elastomeric material and the compression can be adjusted by a change in the elastomeric material.

34. The device of 33, wherein the spring element comprises a leaf spring, a coil spring, an elastomeric material, a wave spring, or a Belleville washer.

35. The device of 34, wherein the elastic member comprises a metal, metal alloy or elastomer material.

36. The device of 21, further comprising an extramedullary bone compression device operatively configured to engage with the intermedullary compression device.

37. The device of 29, wherein the force applied by the compression mechanism on the fixation members can be adjusted in use by adjusting the distance along the longitudinal axis between the anchor point on the external sleeve and the anchor point on the rod.

38. The device of 37, wherein the distance along the longitudinal axis between the anchor point on the external sleeve can be adjusted by engaging a threaded anchor mount.

39. A method of providing continuous bone compression for bone healing comprising the step of: inserting the device of 21 into the medullary canal of a bone segment.

40. The method of 36, wherein the amount of compression applied to the bone segment is patient specific.

41. The method of 36, wherein the bone segment comprises a mid-foot, a hind foot, a toe or finger phalange, a lumbar spine, a pelvis, a hip, a femur, a tibia, an ankle, or a wrist.

48. A method of providing continuous bone compression for bone healing comprising the step of: providing the device of 42 onto a bone segment in an exoskeletal arrangement.

49. The method of 48, wherein the amount of compression applied to the bone segment is tuned by providing a pre-selected degree of elasticity in the elastic loop.

51. A two part screw member having a first threaded portion having a portion having a torque driving surface at a proximal end, and a central axial cannulation and second head screw portion which also has a central cannulation and a torque driving surface which cooperates with the torque driving surface of the first threaded portion and an elastic element which is captured in the central cannulation.

While certain representative embodiments and details have been shown for the purpose of illustrating the subject invention, it will be apparent to those skilled in this art that various changes and modifications can be made therein without departing from the scope of the subject invention. In this regard, the scope of the invention is to be limited only by the following claims.

What is claimed is:

1. An orthopedic implant having continuously tunable variable compression for a bone or bone segment comprising:
a construct defining an axis and having a first member with at least one first member fixation structure capable of fixing the first member to the bone segment, and a second member with at least one second member fixation structure capable of fixing the second member to the bone segment; and
a compression mechanism operatively coupled to the first member and to the second member and capable of applying a force to the first member or the second member in the direction of the axis and having a control that adjusts the degree of force by means of the rotation of an associated element.

2. An orthopedic implant as set forth in claim 1, wherein the first member fixation structure is an aperture in the first member and a fixation member that is received in the aperture.

3. An orthopedic implant as set forth in claim 2, wherein the fixation member is a screw, nail or pin.

4. An orthopedic implant as set forth in claim 2, wherein the second member fixation structure is an aperture in the first member and a fixation member that is received in the aperture.

5. An orthopedic implant as set forth in claim 4, wherein the fixation member is a screw, nail or pin.

6. An orthopedic implant as set forth in claim 1, wherein the compression mechanism is an elastic element.

7. An orthopedic implant as set forth in claim 6, wherein the elastic element is an elastic cable, an elastic loop, a spring, or an elastic ring.

8. An orthopedic implant as set forth in claim 6, wherein the compression mechanism comprises a telescoping relationship between the first member and the second member.

9. An orthopedic implant as set forth in claim 8, wherein the control is a cam or a threaded member.

10. An orthopedic implant as set forth in claim 1, wherein the first member fixation member is a thread or a barb.

11. An orthopedic implant as set forth in claim 1, wherein the construct is an intermedullary implant or a screw.

12. An orthopedic implant as set forth in claim 11 wherein the intermedullary implant or screw has a round cross section.

13. An orthopedic implant as set forth in claim 12, wherein the intermedullary implant comprises an external sleeve having a central opening and the compression mechanism comprises a rod that resides in the central opening and which is operatively joined to an elastic member that bears against the rod in the direction of the axis.

14-41. (canceled)

42. An orthopedic compression device for use with a bone or bone segment, comprising:
a) at least one plate assembly comprising a first plate member that has a channel defining three planes that retain and form a bearing surface for a second plate member so as to form a telescoping relationship along an axis with the first plate member, and the first plate member, and the second plate member each have an aperture that receives a first fixation member and a second fixation member respectively; and
(b) an elastomeric or metal member which engages the first plate member and the second plate member so as to apply a compressive force symmetrically in the direction of to the axis and at the aperture of the first plate member and the second plate member which is transmitted to the first fixation member and the second fixation member.

43. The device of claim 42, wherein the relationship between the first plate member and the second plate member is a dovetail relationship.

44. The device of claim 42, wherein the elastomeric member is an elastic loop.

45. The device of claim 44, wherein the elastic loop is seated in a groove.

46. The device of claim 45, wherein the groove extends around an outer circumference of the plate assembly or is in a top or bottom surface of the first plate member and a top or bottom surface of the second plate member.

47. The device of claim 45, wherein the elastic loop is positioned between the first plate member and the second plate member and engages a retaining member on each.

48-49. (canceled)

50. A orthopedic compression device having a long axis and a first externally threaded member which is in a variable length axial alignment with a second member having a torque driving surface and the first member is capable of a rotational cooperation about the axis with the second member and an elastic element which is capable of exerting a force in the direction of the axis on the first or the second member.

51. (canceled)
